(19) United States
(12) Patent Application Publication
(10) Pub. No.: US 2020/0319279 A1
(43) Pub. Date: Oct. 8, 2020

(54) TECHNIQUES FOR DEUTERIUM METABOLIC IMAGING AND RELATED SYSTEMS AND METHODS

(71) Applicant: Yale University, New Haven, CT (US)

(72) Inventors: Henk De Feyter, West Haven, CT (US); Robin De Graaf, Hamden, CT (US)

(73) Assignee: Yale University, New Haven, CT (US)

(21) Appl. No.: 16/955,750

(22) PCT Filed: Dec. 21, 2018
(86) PCT No.: PCT/US2018/067089

§ 371 Date: Jun. 18, 2020

Related U.S. Application Data
(60) Provisional application No. 62/608,861, filed on Dec. 21, 2017.

(51) Int. Cl. G01R 33/485; G01R 33/48; A61B 5/055; A61B 5/00

(52) U.S. Cl. CPC G01R 33/485; G01R 33/4822; A61B 5/055; A61B 2576/026; A61B 5/4088; A61B 5/4094; A61B 5/4842; A61B 5/0042

(57) ABSTRACT

Techniques for deuterium metabolic imaging (DMI) are provided. According to some aspects, metabolic imaging techniques are described in which deuterium (i.e., 2H)-labeled molecules are detected to assess metabolic processes. These techniques may have an improved spatial resolution compared to conventional techniques, and may provide the ability to determine metabolic information on a voxel-by-voxel basis to form a metabolic image of an imaged volume.

### CROSS-REFERENCE OF RELATED APPLICATIONS

This application is a national stage filing under 35 U.S.C. § 371 of International Patent Application Serial No. PCT/US2018/067089, filed Dec. 21, 2018, which claims priority to U.S. Application Ser. No. 62/608,861, filed Dec. 21, 2017. The contents of these applications are incorporated herein by reference in their entirety.

### GOVERNMENT SUPPORT

This invention was made with government support under NS087568 awarded by the National Institutes of Health. The government has certain rights in the invention.

### BACKGROUND

Metabolic dysfunction plays a major role in the pathogenesis of many neurological diseases, including brain tumors, epilepsy, traumatic brain injury and multiple sclerosis. As a result, clinicians may wish to image the metabolism of a subject in-vivo to diagnose or monitor progression of a disease.

One approach to in-vivo metabolic imaging is based on positron emission tomography (PET) which detects glucose uptake by imaging radioactively-labeled substances introduced into the body. Another approach is to use magnetic resonance imaging (MRI) to detect nutrients or substrates that are labeled with ¹³C.

### SUMMARY

According to some aspects, a magnetic resonance imaging system is provided comprising at least one processor configured to instruct a magnetic resonance imager to acquire a deuterium-labeled three-dimensional (3D) data set of at least part of a subject, determine a spectrum for each of a plurality of voxels of the 3D data set, at least a portion of the determined spectra comprising at least one peak corresponding to a deuterium-labeled molecule, and generate at least one metabolic image based at least in part on the spectra determined for the voxels in the 3D dataset.

According to some embodiments, the at least one processor is configured to acquire the deuterium-labeled 3D data set by instructing the magnetic resonance imager to apply one or more magnetic pulses to the at least part of the subject.

According to some embodiments, the spectra determined for the voxels in the 3D dataset are determined based on deuterium-labeled 3D data acquired from a single magnetic pulse.

According to some embodiments, the at least one peak corresponding to a deuterium-labeled molecule comprises a peak corresponding to 2H-labeled glucose, 2H-labeled lactate, 2H-labeled glutamine, or 2H-labeled glutamate.

According to some embodiments, the at least one processor is configured to quantify intensities of a plurality of peaks within the determined spectrum.

According to some embodiments, the plurality of peaks include a peak that corresponds to 2H-labeled glucose and a peak that corresponds to 2H-labeled lactate.

According to some embodiments, generating the at least one metabolic image comprises generating at least one color-coded image that reflects an amount of metabolic activity for one or more deuterium-labeled molecules at different voxel locations in a 3D volume.

According to some embodiments, the at least one processor is further configured to produce a plurality of metabolic images based on respective deuterium-labeled 3D data sets acquired at different times.

According to some embodiments, the at least one processor is further configured to determine a glucose uptake rate and/or an acetate uptake rate.

According to some embodiments, the at least one metabolic image has a spatial resolution of less than 10 mL.

According to some embodiments, the magnetic resonance imaging system further comprises at least one computer readable medium comprising instructions, and wherein the at least one processor is configured to perform said acts of instructing, determining and generating by executing the instructions.

According to some embodiments, the plurality of voxels of the 3D data set are arranged in a single layer of voxels, thereby representing a two-dimensional slice.

According to some aspects, a computer-implemented method of metabolic imaging is provided, the method comprising acquiring a deuterium-labeled three-dimensional (3D) data set of at least part of a subject, determining, using at least one processor, a spectrum for each of a plurality of voxels of the 3D data set, at least a portion of the determined spectra comprising at least one peak corresponding to a deuterium-labeled molecule, and generating, using the at least one processor, at least one metabolic image based at least in part on the spectra determined for the voxels in the 3D dataset.

According to some embodiments, acquiring the deuterium-labeled 3D data set comprises instructing a magnetic resonance imager to apply one or more magnetic pulses to the at least part of the subject.

According to some embodiments, the at least one peak corresponding to a deuterium-labeled molecule comprises a peak corresponding to 2H-labeled glucose, 2H-labeled lactate, 2H-labeled glutamine, or 2H-labeled glutamate.

According to some embodiments, the method further comprises quantifying, using the at least one processor, intensities of a plurality of peaks within the determined spectrum.

According to some embodiments, the plurality of peaks include a peak that corresponds to 2H-labeled glucose and a peak that corresponds to 2H-labeled lactate.

According to some embodiments, generating the at least one metabolic image comprises generating at least one color-coded image that reflects an amount of metabolic activity for one or more deuterium-labeled molecules at different voxel locations in a 3D volume.

According to some embodiments, the method further comprises determining, using the at least one processor, a glucose uptake rate and/or an acetate uptake rate.

According to some aspects, at least one computer readable medium is provided comprising instructions that, when executed by at least one processor, perform a method of metabolic imaging, the method comprising acquiring a deuterium-labeled three-dimensional (3D) data set of at least part of a subject, determining, using at least one processor, a spectrum for each of a plurality of voxels of the 3D data set, at least a portion of the determined spectra comprising at least one peak corresponding to a deuterium-labeled molecule, and generating, using the at least one processor, at least one metabolic image based at least in part on the spectra determined for the voxels in the 3D dataset.

According to some embodiments, the at least one peak corresponding to a deuterium-labeled molecule comprises a peak corresponding to 2H-labeled glucose, 2H-labeled lactate, 2H-labeled glutamine, or 2H-labeled glutamate.

According to some embodiments, generating the at least one metabolic image comprises generating at least one color-coded image that reflects an amount of metabolic activity for one or more deuterium-labeled molecules at different voxel locations in a 3D volume.

The foregoing apparatus and method embodiments may be implemented with any suitable combination of aspects, features, and acts described above or in further detail below. These and other aspects, embodiments, and features of the present teachings can be more fully understood from the following description in conjunction with the accompanying drawings.

### BRIEF DESCRIPTION OF DRAWINGS

Various aspects and embodiments will be described with reference to the following figures. It should be appreciated that the figures are not necessarily drawn to scale. In the drawings, each identical or nearly identical component that is illustrated in various figures is represented by a like numeral. For purposes of clarity, not every component may be labeled in every drawing.

FIG. 1 is a flowchart of a method of generating metabolic images through deuterium metabolic imaging, according to some embodiments;

FIG. 2 is a schematic illustrating metabolic processes of deuterium-labeled glucose and acetate, according to some embodiments;

FIGS. 3A and 3B are deuterium NMR spectra illustrating comparative amounts of deuterium-labeled substances in a rat brain, according to some embodiments;

FIG. 4A depicts a DMI visualization of the Warburg effect in a human subject with glioblastoma, according to some embodiments;

FIG. 4B depicts deuterium NMR spectra at four locations within a human subject with glioblastoma corresponding to locations identified in FIG. 4A, according to some embodiments;

FIGS. 5A-5B depict illustrative NMR spectra of a human subject's brain following oral administration of glucose to the subject at two different magnetic field strengths, according to some embodiments;

FIG. 6 is a diagram of an illustrative MRI system suitable for practicing some embodiments of the present disclosure; and

FIG. 7 illustrates an example of a computing system environment on which aspects of the invention may be implemented.

### DETAILED DESCRIPTION

A wide range of neurodegenerative diseases may be characterized by metabolic components that play a role at various stages of pathogenesis. For instance, deficient glucose metabolism (“hypometabolism”) due to mitochondrial dysfunction is considered a common factor in the development of Alzheimer's and Parkinson's diseases in addition to acquired epilepsy. As another example, secondary brain injury acquired after sudden head trauma has been described to disturb homeostasis of essential neural metabolites and neurochemicals.

Some conventional nuclear magnetic resonance (NMR) methods that detect metabolism rely on the use of nutrients or substrates that are labeled with the stable isotope ¹³C. However, such data can be acquired only from relatively large volumes because of the poor sensitivity of ¹³C detection. Because of the large volumes required for ¹³C MR techniques, it is impractical to generate images using such techniques.

Other challenges associated with conventional metabolic imaging techniques based on ¹³C magnetic resonance spectroscopy (MRS) include the requirement to perform lipid suppression and broadband decoupling. Hyperpolarized ¹³C MRS requires expensive equipment for hyperpolarization, has a short time window for signal detection, and is currently limited to a single clinically-approved substrate, pyruvate. These requirements have largely prevented ¹³C MRS from maturing into a clinically-viable method of metabolic imaging.

Other in-vivo metabolic imaging techniques include the use of positron emission tomography (PET) to detect glucose uptake by imaging radioactively-labeled substances introduced into the body. Although the spatial resolution and sensitivity of PET is acceptable, such techniques may have a low clinical adoption rate to assess metabolic processes due to the requirement to use radioactively-labeled substances and the limited metabolic information that can be obtained using such techniques. For example, typically only the uptake of a glucose analog (e.g., ¹⁸F-deoxyglucose or ¹⁸FDG) can be interrogated using PET, which does not provide information about downstream glucose metabolism.

The inventors have recognized a need for non-invasive metabolic imaging techniques that do not require the administration of radioactively-labeled substances to a patient (e.g., as required for PET) and provide an alternative to conventional ¹³C NMR imaging, which has intrinsically low sensitivity.

The inventors have recognized and appreciated metabolic imaging techniques in which deuterium (i.e., ²H)-labeled molecules are detected to assess metabolic processes. These techniques may have an improved spatial resolution compared to ¹³C MR techniques, providing the ability to determine metabolic information on a voxel-by-voxel basis to form a metabolic image of an imaged volume. The metabolic imaging techniques in accordance with some embodiments are referred to herein as “deuterium metabolic imaging” (DMI), representing the fact that peaks of deuterium-labeled molecules in individual voxels are determined based on ²H nuclear magnetic resonance (NMR).

In some embodiments, deuterium metabolic imaging techniques may perform NMR imaging (also referred to herein as Magnetic Resonance Imaging, or MRI) of a deuterium-labeled substrate introduced into a subject. In some cases, such substrates may require minimal pharmaceutical preparation for intravenous infusion in human subjects. For instance, ²H-labeled nutrients such as glucose, acetate and ketone bodies may not be subject to drug approval and may be purchased pre-tested for sterility, and at low cost.

In some embodiments, deuterium metabolic imaging techniques may perform MRI of a substance produced within a subject as a result of metabolic processes that act upon a deuterium-labeled substrate introduced into the subject. Such substances may include metabolites and/or intermediates in metabolic pathways. For instance, deuterium-labeled glucose introduced into a subject may produce pyruvate and lactate as a result of the deuterium-labeled glucose being converted in the glycolytic pathway. As a result of such a process, the resultant pyruvate and/or lactate may be deuterium-labeled and may be imaged through the deuterium metabolic imaging techniques described herein.

In some embodiments, deuterium metabolic imaging techniques may produce an MRI spectrum for each of a plurality of voxels within a three-dimensional (3D) volume. Imaging of a deuterium-labeled substrate introduced into a subject within each of a plurality of voxels may allow generation of a 3D map of a metabolite within the subject. In cases where the substrate produces additional deuterium-labeled substances as a result of metabolic processes, 3D maps of those substances may be generated as well.

In some embodiments, by combining multiple 3D maps of deuterium-labeled substances, an understanding of the metabolic processes occurring within the 3D volume may be obtained. For example, as discussed above, deficient glucose metabolism is considered a factor in the development of Alzheimer's and Parkinson's diseases. Imaging of deuterium-labeled glucose (and/or other deuterium-labeled substances) within a subject may thereby allow identification of such diseases and/or allow the progression of such diseases to be tracked.

Following below are more detailed descriptions of various concepts related to, and embodiments of, techniques of deuterium metabolic imaging. It should be appreciated that various aspects described herein may be implemented in any of numerous ways. Examples of specific implementations are provided herein for illustrative purposes only. In addition, the various aspects described in the embodiments below may be used alone or in any combination, and are not limited to the combinations explicitly described herein.

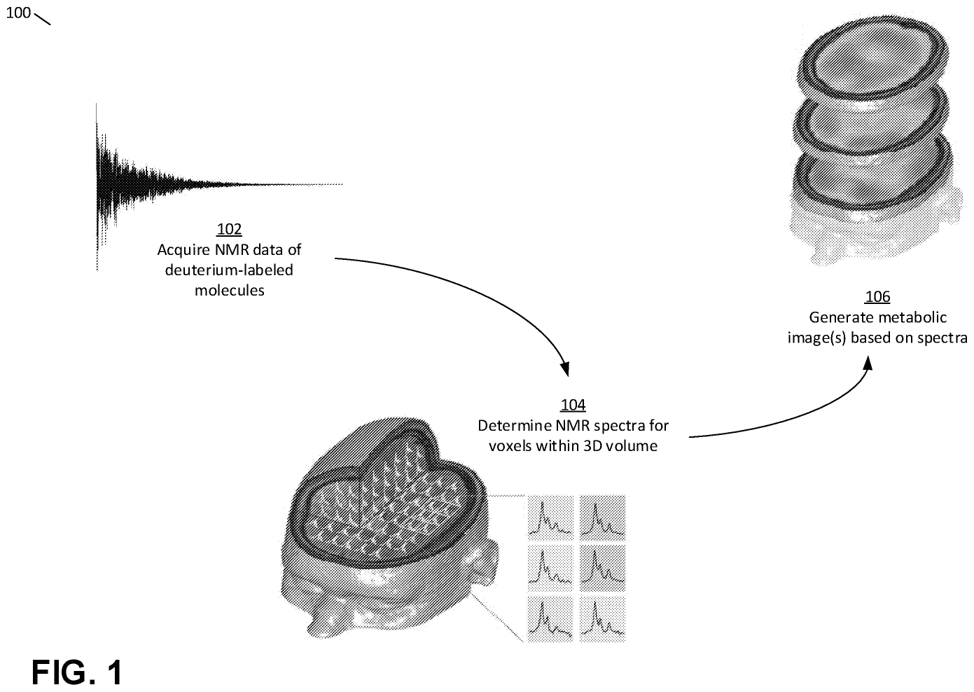

FIG. 1 is a flowchart of a method of generating metabolic images through deuterium metabolic imaging, according to some embodiments. Method 100 may be performed by a suitably configured MRI system or device, examples of which are described below.

In act 102, the MRI system or device performing method 100 acquires NMR data of deuterium-labeled molecules. Act 102 may comprise performing one or more NMR scans of any suitable subject, including a human subject, and may comprise one or more NMR scans of particular regions of the subject's body. In some cases, the regions of the body may be selected based on particular organs of interest. For example, the subject's head may be scanned to produce NMR data of the subject's brain. Other suitable organs that may be scanned in act 102 include the liver and the heart. References to a “scan” or “scanning” via NMR herein refer simply to the performance of any suitable number of NMR sequences in which oscillating magnetic pulses are applied to a target area.

In the example of FIG. 1, a subject scanned in act 102 comprises one or more deuterium-labeled substances and scanning operations performed in act 102 may produce NMR data corresponding to any one or more of these substances. In some cases, one or more of the deuterium-labeled substances scanned in act 102 was previously administrated to the subject. Additionally, or alternatively, one or more of the deuterium-labeled substances scanned in act 102 includes one or more substances produced within the subject's body from molecules of a previously administrated deuterium-labeled substrate, which thereby themselves contain deuterium.

In some embodiments, a subject scanned in act 102 may have previously been administered one or more deuterium-labeled substrates. Such substrates may be administered to the subject in any suitable manner, including oral administration and/or via infusion (which may include intravenous administration in addition to other non-oral routes such as intramuscular injections or epidural routes). Act 102 may be performed a suitable time after administration of such substrates (e.g., between 50 and 115 minutes following administration in the case of oral administration of [6, 6′-²H₂]-glucose), immediately after administration of one or more substrates, or during administration of one or more substrates.

According to some embodiments, one or more deuterium-labeled substrates administered to a subject scanned in act 102 may comprise deuterium-labeled organic molecules. For instance, the one or more deuterium-labeled substrates may include one or more deuterium-labeled sugars (e.g., glucose), deuterium-labeled amino acids (e.g., glycine), deuterium-labeled ketones, deuterium-labeled salts (e.g., acetate), deuterium-labeled glutamate, or combinations thereof.

In some embodiments, the MRI system or device performing method 100 comprises one or more RF coils tuned for NMR measurement of deuterium. In some embodiments, the MRI system or device may also comprise RF coils tuned for NMR measurement of protons. In some cases, the MRI system or device performing method 100 may be configured to acquire NMR data of a number of different deuterium-labeled molecules with a single pulse.

Deuterium is a quadrupolar nucleus with spin 1 and a small gyromagnetic ratio. Deuterated molecules (i.e., molecules containing one or more deuterium atoms) generally have a much shorter NMR T1 relaxation time than equivalent molecules labeled with ¹³C. It has been found by the inventors, however, that T2 values of deuterium labeled metabolites are relatively long, which results in well resolved ²H spectra of substances such as deuterated glucose, glutamate and lactate in vivo, which allows for the quantitation of multiple such substances with a single RF pulse. Further, producing a deuterium MRI spectrum requires comparatively low RF power. This, coupled with the molecules low Larmor frequency allows for rapid pulsing (e.g., <500 ms) for extended signal averaging.

According to some embodiments, in act 102 the MRI system or device performing method 100 may acquire NMR data for a plurality of volume elements (“voxels”) within a selected volume. For instance, NMR data may be acquired for a plurality of voxels at different locations within a human brain. Voxels may be of any suitable size, such as between 50 mm³ and 1 cm³. Voxel positions for acquired data may be determined via any suitable method, for instance via detecting differences in frequency, phase and/or signal timing within the acquired data. In some embodiments, the MRI system or device performing method 100 may, in act 102, apply a magnetic field configured to allow subsequent position detection of signals received from different voxels. For example, a magnetic field gradient may be applied in act 102 to alter the primary magnetic field in a spatially specific pattern (e.g., via the use of gradient coils), thereby allowing position detection through detecting spatial variation in the Larmor frequency.

In act 104 of method 100, NMR spectra are determined for a plurality of voxels within a 3D volume based on at least some of the NMR data acquired in act 102. As discussed above, NMR data acquired in act 102 may be analyzed to determine signals produced by each of a plurality of different voxels. In act 104, such signals may be analyzed to produce corresponding NMR spectra for at least some of these voxels. As referred to herein, an NMR spectrum indicates relative signals strengths of acquired NMR data as a function of chemical shift, which is a more convenient way to represent the resonant frequency shift. The chemical shift is not dependent on the magnetic field strength of the device which produced the data and is typically expressed as a parts per million (ppm) deviation from the resonant frequency of a reference substance (e.g., tetramethylsilane). As such, in some embodiments, a result of act 104 may be a plurality of spectra each indicating relative signal strengths at a plurality of different chemical shifts.

In some embodiments, act 104 may comprise determining NMR spectra at a plurality of different times. In such cases, act 102 may also comprise acquiring NMR data at a plurality of different times, such as by applying a sequence of single pulses that each produce ²H NMR data corresponding to multiple different deuterated molecules. In some cases, observation of NMR spectra over time may provide insight into changes in a subject's metabolism over a time scale of seconds or minutes. In some embodiments, act 104 (and/or act 106) may comprise performing a spectral fit of one or more spectral lines at known resonant shifts (or chemical shifts) to the experimentally obtained spectral data to determine intensities of the one or more spectral lines.

In the example of FIG. 1, in act 106 the MRI system or device performing method 100 may examine the intensity of selected peaks within the NMR spectra determined in act 104 to produce one or more area and/or volume density maps of one or more substances associated with the chemical shift values of selected peaks. As will be discussed further below, the inventors have recognized techniques to resolve the chemical shifts of various deuterated molecules that play a role in metabolism. As such, density maps, whether produced for an area (e.g., a slice of the 3D volume) or for part or all of the 3D volume, can provide insight into metabolism in various locations within the scanned volume. The aforementioned density maps are referred to herein as metabolic images.

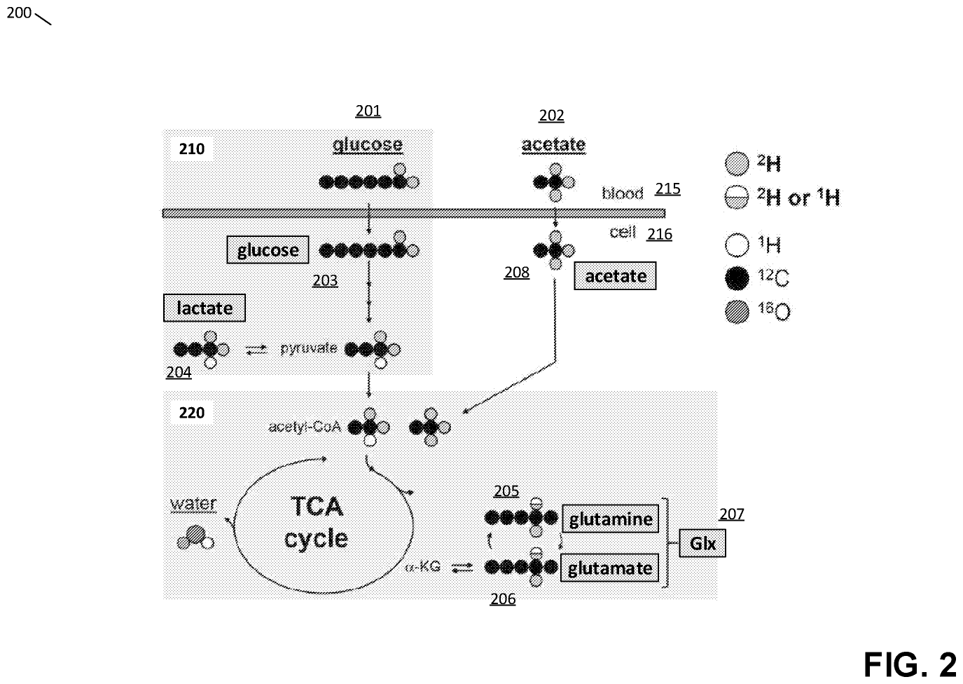

FIG. 2 is a schematic illustrating metabolic processes of deuterium-labeled glucose and acetate, according to some embodiments. Illustrative schematic 200 depicts metabolism for a particular deuterated form of the glucose molecule, namely [6, 6′-²H₂]-glucose 201. In addition, schematic 200 depicts metabolism for a particular deuterated form of acetate, namely [²H₃]-acetate 202. The locations of relevant ²H and ¹H atoms within the molecules shown in the example of FIG. 2 are identified with light grey circles and half-filled grey circles, respectively. For the purposes of FIG. 2, the pathways are illustrated for deuterium-labeled substrates glucose and acetate (201 and 202, respectively) that are initially within the subject's blood 215. Subsequent to their disposition into the blood 215, at least some of the deuterated substrate molecules enter the cell 216.

As FIG. 2 illustrates, after entering the cell, glucose 203 within the cell may be metabolized in the glycolytic pathway (identified by shaded region 210 in FIG. 2), which has pyruvate as the end product. Pyruvate can be converted to lactate 204, or can enter the mitochondria for complete oxidation (identified by shaded region 220 in FIG. 2) in the tricarboxylic acid (TCA) cycle. Both oxidation of glucose and acetate may result in labeling of glutamine and glutamate through rapid exchange with the TCA-cycle intermediate a-ketoglutarate (a-KG). During formation of citrate, one or more protons of the acetyl-CoA methyl group may be removed. In the case of ²H labeled acetyl-CoA the citrate formation can therefore result in loss of ²H label, illustrated in FIG. 2 by the half-filled circles in glutamate 205 and glutamine 206. The label loss during citrate formation may give rise to two different labeled species of glutamine and glutamate, either having with one or two ²H atoms: [4, 4′-²H₂] and [4-²H]. The combined abundances of glutamine and glutamate are referred to herein as Glx (labeled 207).

In the case of acetate substrate 202, acetate 208 within the cell and oxidation of the acetate to produce glutamate 205 and glutamine 206 (Glx) via oxidation may be quantified.

As a result of the above-described process, deuterated glucose and/or acetate in the blood can give rise to deuterated glucose, lactate, glutamine and glutamate in the cells. Via the DMI techniques described herein, NMR spectra of one or more of these deuterium-labeled substances may be detected and quantified, thereby providing insight into the metabolic processes of a subject.

For instance, in the example of FIG. 2, a measure of glycolytic metabolism and/or oxidative metabolism may be determined based on the relative abundances of NMR spectral lines corresponding to one or more of glucose, acetate, lactate, glutamine and glutamate. That is, activity along the glycolic pathway that leads to pyruvate and lactate may be quantified by measuring an amount of lactate in an NMR spectrum, and activity along the oxidative pathway that leads to glutamine and glutamate may be quantified by measuring an amount of glutamate and/or glutamine in the NMR spectrum. Such an analysis may, in some cases, allow for detection of cancer cells via the Warburg effect, which is the observation that cancer cells tend to favor metabolism via glycolysis rather than oxidation, whereas most other cells in the body favor oxidation.

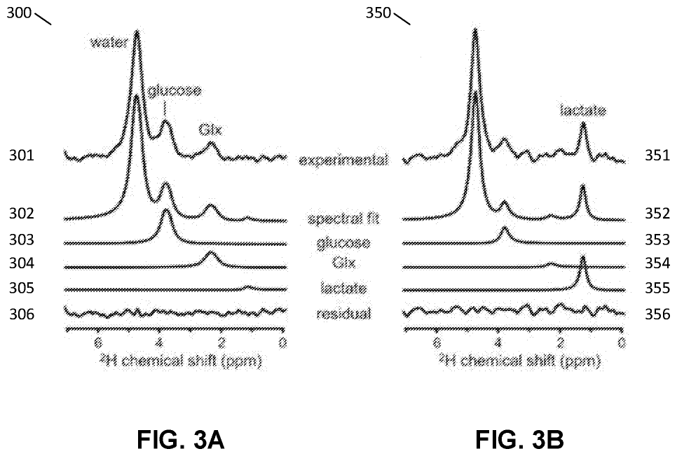

FIGS. 3A and 3B are deuterium NMR spectra illustrating comparative amounts of deuterium-labeled substances in a rat brain, according to some embodiments. Spectra 300 and 350 shown in FIGS. 3A and 3B, respectively, depict NMR spectra as described above, with an intensity of the resonant signal (vertical axis) at various different ²H chemical shift values (horizontal axis) illustrated.

In the example of FIGS. 3A and 3B, NMR spectra produced from data acquired from a subject is shown as spectra 301 and 351, respectively. To determine the contributions to these spectra from varying amounts of glucose, Glx and lactate, computed spectra for each of these individual ²H-labeled substances are shown in spectra 303, 304 and 305, respectively, for FIG. 3A and in spectra 353, 354 and 355, respectively, for FIG. 3B. The residuals of the spectra 301 and 351 after subtraction of spectra 303-305 or 353-355, respectively, are shown as spectra 306 and 356. In some embodiments, spectra 302-305 and 352-355 may be determined via a processing of spectral fitting.

In the example of FIGS. 3A-3B, the data acquired to produce spectra 300 was obtained from a normal-appearing rat brain, whereas the data acquired to produce spectra 350 was obtained from a portion of a rat brain containing a tumor lesion. Both sets of spectra 300 and 350 were produced from NMR data acquired from a single voxel having a size of 2 mm×2 mm×2 mm. As discussed above, the data represented by either spectra 300 or spectra 350 could be produced from a single NMR pulse applied to a corresponding subject, since the various peaks in the spectra may be resolvable from NMR data acquired from the single pulse. In the case of glutamine and glutamate, in at least some cases the individual peaks may not be resolvable and consequently a single Glx peak may be observed.

As can be seen in FIGS. 3A and 3B, the ratios of lactate to Glx differ between the normal-looking brain represented by FIG. 3A and the brain including a tumor lesion represented by FIG. 3B. Specifically, the difference in this ratio illustrates the aforementioned Warburg effect, since in the case of a tumor the lactate/Glx value is comparatively high (higher lactate, lower Glx in spectrum 351) and in the case of the normal-looking brain the lactate/Glx value is comparatively low (lower lactate, higher Glx in spectrum 301).

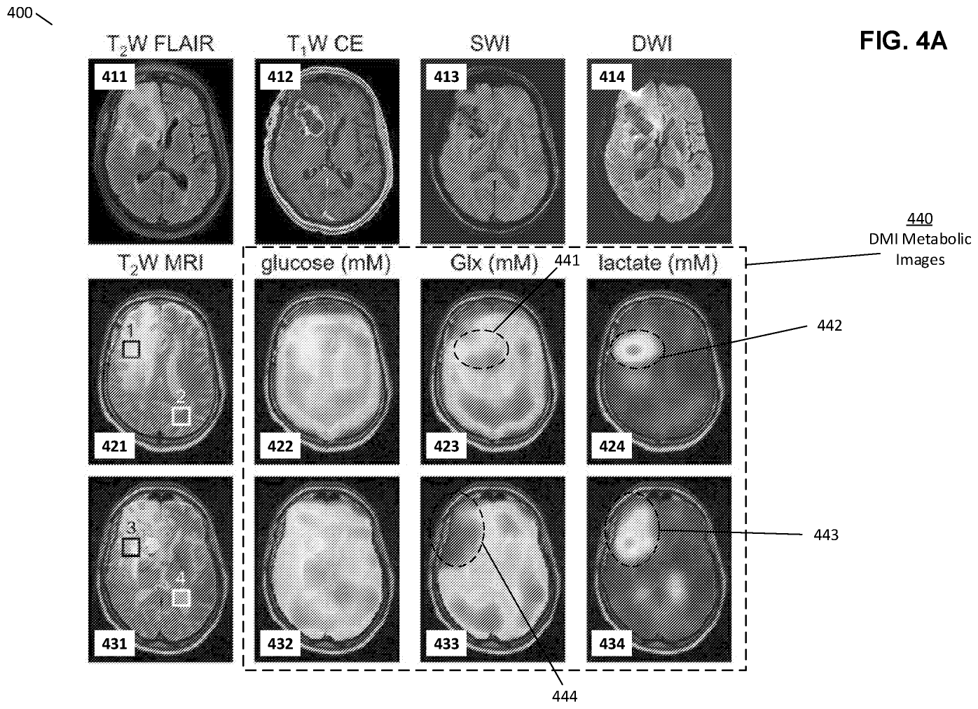

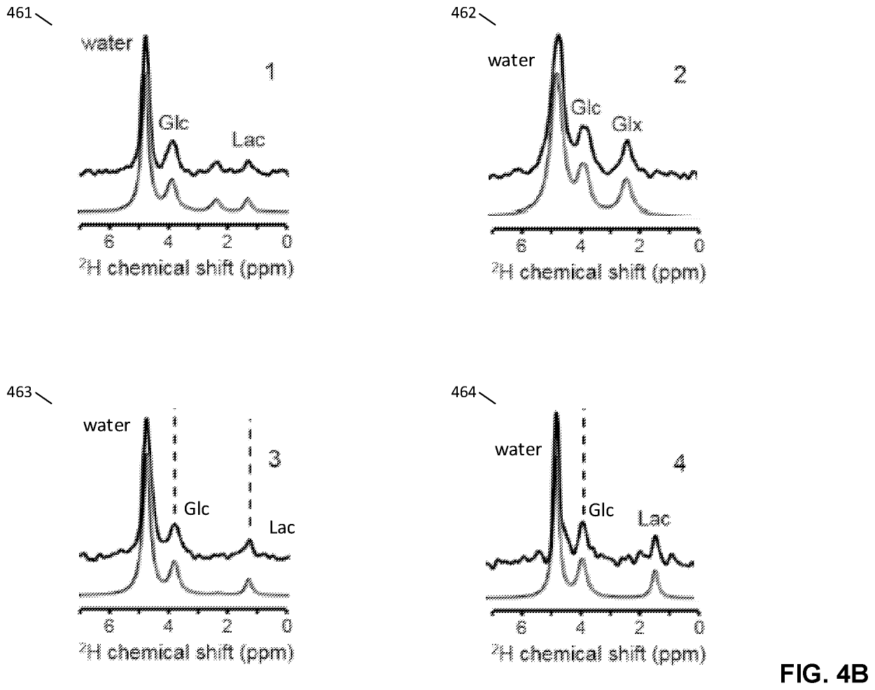

FIG. 4A depicts a DMI visualization of the Warburg effect in a human subject with glioblastoma, according to some embodiments. The images 411-414, 421-424 and 431-434 are images produced from various different MRI techniques, including both conventional MRI techniques and DMI. The images 422-424 and 432-434 (labeled as a group 440) are metabolic images produced through the DMI techniques described herein.

In the example of FIG. 4A, several conventional MRI images 411, 412, 413 and 414 are shown along the top row, representing T2-weighted-Fluid-Attenuated Inversion Recovery (T2-W FLAIR), T1-weighted Contrast Enhanced (T1W CE), Susceptibility Weighted Imaging (SWI) and Diffusion Weighted Imaging (DWI), respectively.

Images 421 and 431 represent conventional T2-weighted MRI images with each being an image of a different slice within the subject's brain. A glioblastoma is located at the upper left of each image, labeled “1” in image 421 and “3” in image 431. Locations 2 and 4 in images 421 and 431 are comparative locations.

According to some embodiments, DMI metabolic images 440 may have been produced via, for instance, the method 100 of FIG. 1 described above. For example, NMR data may have been acquired from the subject and NMR spectra within a plurality of voxels generated. The metabolic images 440 may then be produced by determining the intensity of peaks corresponding to glucose, Glx and lactate within a plurality of voxels within each of a plurality of slices (e.g., a plane of voxels) within the brain, and selecting the brightness (and/or color, not shown in FIG. 4A) of pixels in the image based on the intensity at that location. In the example of FIG. 4A, a brighter pixel generally corresponds to a higher measured intensity.

The Warburg effect may be seen in the example of FIG. 4A, wherein regions 441 and 442 that correspond to location 1 in image 421 show higher lactate activity (bright spot) and lower Glx activity (darker spot). Similarly, in the other slice, regions 443 and 444 that correspond to location 3 in image 431 show higher lactate activity (bright spot) and lower Glx activity (darker spot). FIG. 4A accordingly provides one example in which cancerous cells may be identified from DMI metabolic images and thereby provide a technique for diagnosis.

NMR spectra corresponding to the locations 1-4 of images 421 and 431 are shown in FIG. 4B. Spectra 461, 462, 463 and 464 depicts NMR spectra for locations 1, 2, 3 and 4, respectively, with the darker spectral line representing raw experimental data and the lighter spectral line representing contributions of water, glucose (abbreviated “Glc” in FIG. 4B), lactate (abbreviated “Lac” in FIG. 4B) and Glx produced by spectral fitting. As noted above, locations 1 and 3 which correspond to a location of a glioblastoma exhibit the Warburg effect by exhibiting a comparatively higher lactate activity and a comparatively lower Glx activity (e.g., negligible in spectrum 463). It may be noted that location 4 shows an indication of higher lactate. Location 4 is situated within a ventricle and mainly contains cerebrospinal fluid (CSF). The higher lactate measurement at location 4 is likely because the lactate has leaked out of the glioblastoma and into the CSF.

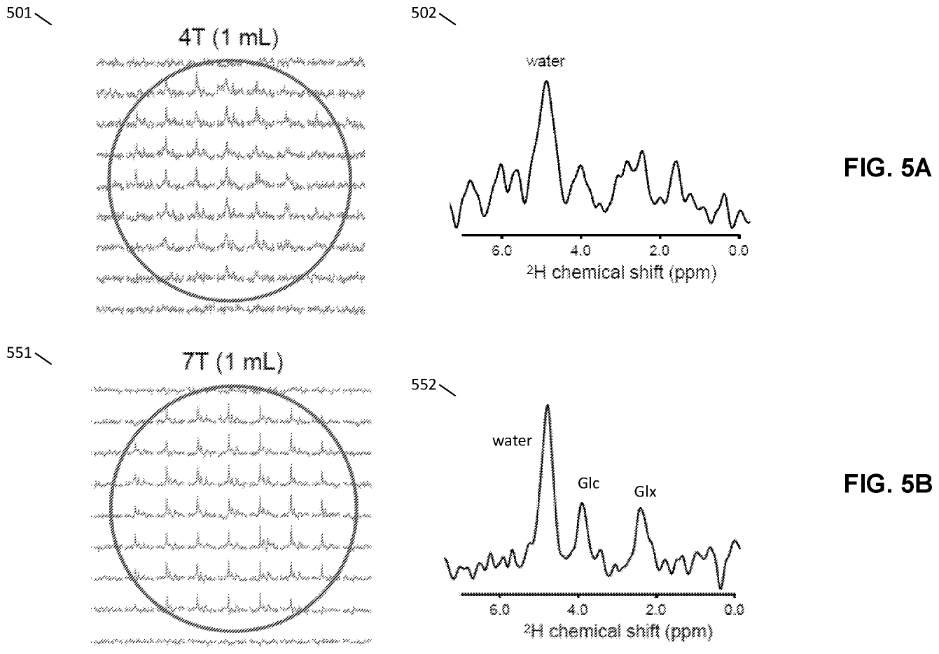

FIGS. 5A-5B depict illustrative NMR spectra of a human subject's brain following oral administration of glucose to the subject at two different magnetic field strengths, according to some embodiments. It has been observed by the inventors that the sensitivity of deuterium metabolic imaging has a strong dependence on the magnetic field strength (e.g., near-quadratic in human studies).

FIG. 5A depicts NMR spectra 501 of the subject's brain, representing spectra of several 1 mL voxels (=1 cm³ voxels) captured with an MRI system having a 4 Tesla primary magnet. A representative spectrum 502 selected from amongst the spectra 501 is shown. In comparison, NMR spectra 551 shown in FIG. 5B represent spectra of the subject's brain (again with 1 mL voxels) captured with a different MRI system having a 7 Tesla primary magnet. A representative spectrum 552 selected from amongst the spectra 551 is shown.

It will be noted from FIGS. 5A and 5B that spectrum 552 has a much higher spatial resolution than spectrum 551 (an increase in signal to noise of around 2.3). This increase is due to the aforementioned non-linear increase in deuterium sensitivity with magnetic field strength. It will also be noted that signal to noise could alternatively (or additionally) be increased by increasing the voxel size. As such, increasing the voxel size to larger than 1 mL with a 4 Telsa primary magnet may also be expected to increase signal to noise of the resultant spectra.

FIGS. 5A and 5B also provide an example of measuring successive DMI spectra at a plurality of times. As shown by spectra 501 and 551, by applying a sequence of pulses, a sequence of respective NMR spectra may be produced, each of which may individually provide an indication of the relative intensities of deuterated molecules.

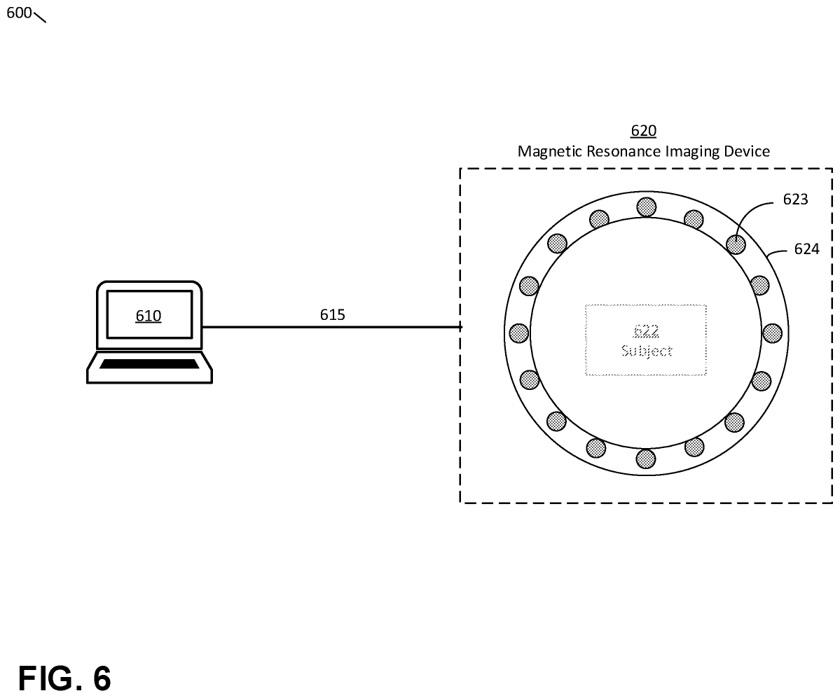

FIG. 6 is a diagram of an illustrative MRI system suitable for practicing some embodiments of the present disclosure. System 600 is provided as an illustrative example of an MRI system which may be operated to perform any of the above-described techniques, including but not limited to one or more acts of method 100 shown in FIG. 1. In the example of FIG. 6, a computing device 610 is coupled to an MRI device 620 via a communication link 615. Optionally, a subject 622 may be placed into the MRI device 620.

According to some embodiments, computing device 610 is configured to execute instructions that, when executed, perform aspects of DMI discussed herein. Such instructions may be implemented in any suitable way, including via software, hardware, or a combination of both.

In some embodiments, computing device 610 is located within a common housing with MRI device 620. For instance, a console of an MRI system that contains the MRI device 620 may provide controls for a user to operate the MRI device, and the computing device 610 may be part of such a console or otherwise coupled to it. In some cases, the computing device 610 may be located close to, but separate from, the MRI device 620 (e.g., in an adjacent room) and configured to communicate with the MRI device via the link 615, which may comprise any suitable wired and/or wireless connections.

In the example of FIG. 6, MRI device 620 includes RF coils 623 and RF shield 624. At least some of the RF coils 623 may be configured (e.g., tuned) to detect deuterium. In some embodiments, MRI device 620 may also comprise RF coils tuned for NMR measurement of protons.

In some embodiments, the RF coils 623 may consist of, or may comprise, one or more transverse electromagnetic (TEM) coils. In some embodiments, the MRI device 620 may comprise a 4-coil phased array which may, in some cases, be operable as a single RF coil. In some embodiments, RF coils 623 may be configured to produce phase-encoding gradients (e.g., may be instructed to produce pulses according by computing device 610, or otherwise). In some embodiments, MRI device 620 may comprise one or more phased array receivers. The RF coils 623 may include any type of volume and/or surface coils, including a combination of volume and surface coils.

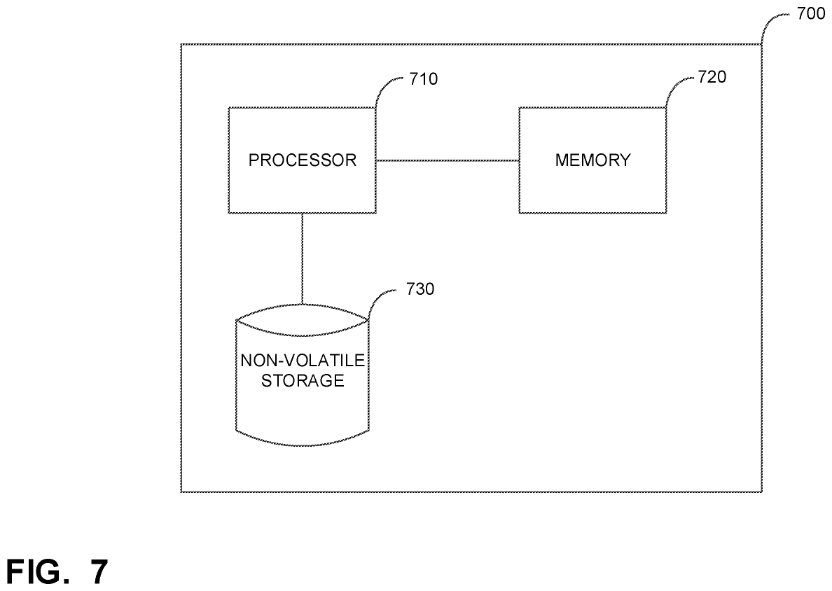

An illustrative implementation of a computer system 700 that may be used to perform any of the aspects of deuterium metabolic imaging is shown in FIG. 7. The computer system 700 may include one or more processors 710 and one or more non-transitory computer-readable storage media (e.g., memory 720 and one or more non-volatile storage media 730). The processor 710 may control writing data to and reading data from the memory 720 and the non-volatile storage device 730 in any suitable manner, as the aspects of the invention described herein are not limited in this respect. To perform functionality and/or techniques described herein, the processor 710 may execute one or more instructions stored in one or more computer-readable storage media (e.g., the memory 720, storage media, etc.), which may serve as non-transitory computer-readable storage media storing instructions for execution by the processor 710.

In connection with techniques described herein, code used to, for example, instruct an MRI device to produce pulses, to detect received magnetic signals from a subject, to decode a location of a voxel based on a signal, generate an NMR spectrum, perform a spectral fit, generate a metabolic image, etc. may be stored on one or more computer-readable storage media of computer system 700. Processor 710 may execute any such code to provide any techniques for deuterium metabolic imaging as described herein. Any other software, programs or instructions described herein may also be stored and executed by computer system 700. It will be appreciated that computer code may be applied to any aspects of methods and techniques described herein. For example, computer code may be applied to interact with an operating system of an MRI device to operate magnetic fields through conventional processes.

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a suitable framework.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more methods, of which examples have been provided. The acts performed as part of a method may be ordered in any suitable way. Accordingly, embodiments may be constructed in which acts are performed in an order different than illustrated, which may include performing some acts simultaneously, even though shown as sequential acts in illustrative embodiments. It will be appreciated that the provided examples of methods are non-limiting and that other inventive methods may be envisioned based on the above disclosure.

For instance, it will be appreciated based on the above that diagnosis of various diseases may be performed through analysis of DMI-produced metabolic images. One example provided above is cancer in the brain, although the techniques described herein may also be applied to detect cancerous cells in other parts of the body, such as in the heart or liver. By detecting molecules that play a role in metabolism, diagnosis of other diseases may also be performed through DMI.

One illustrative method of diagnosis may comprise acts of administering a ²H-labeled substrate to a subject via any suitable method, subsequently performing deuterium metabolic imaging (e.g., method 100) upon one or more regions of the subject's body to produce one or more metabolic images, and diagnosing a disease (e.g., cancer) based on at least one of the one or more metabolic images.

Some actions may be described herein as being taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A magnetic resonance imaging system comprising:
at least one processor configured to:
instruct a magnetic resonance imager to acquire a deuterium-labeled three-dimensional (3D) data set of at least part of a subject;
determine a spectrum for each of a plurality of voxels of the 3D data set, at least a portion of the determined spectra comprising at least one peak corresponding to a deuterium-labeled molecule; and
generate at least one metabolic image based at least in part on the spectra determined for the voxels in the 3D dataset.

2. The magnetic resonance imaging system of claim 1, wherein the at least one processor is configured to acquire the deuterium-labeled 3D data set by instructing the magnetic resonance imager to apply one or more magnetic pulses to the at least part of the subject.

3. The magnetic resonance imaging system of claim 2, wherein the spectra determined for the voxels in the 3D dataset are determined based on deuterium-labeled 3D data acquired from a single magnetic pulse.

4. The magnetic resonance imaging system of claim 1, wherein the at least one peak corresponding to a deuterium-labeled molecule comprises a peak corresponding to ²H-labeled glucose, ²H-labeled lactate, ²H-labeled glutamine, or ²H-labeled glutamate.

5. The magnetic resonance imaging system of claim 1, wherein the at least one processor is configured to quantify intensities of a plurality of peaks within the determined spectrum.

6. The magnetic resonance imaging system of claim 5, wherein the plurality of peaks include a peak that corresponds to ²H-labeled glucose and a peak that corresponds to ²H-labeled lactate.

7. The magnetic resonance imaging system of claim 1, wherein generating the at least one metabolic image comprises generating at least one color-coded image that reflects an amount of metabolic activity for one or more deuterium-labeled molecules at different voxel locations in a 3D volume.

8. The magnetic resonance imaging system of claim 1, wherein the at least one processor is further configured to produce a plurality of metabolic images based on respective deuterium-labeled 3D data sets acquired at different times.

9. The magnetic resonance imaging system of claim 1, wherein the at least one processor is further configured to determine a glucose uptake rate and/or an acetate uptake rate.

10. The magnetic resonance imaging system of claim 1, wherein the at least one metabolic image has a spatial resolution of less than 10 mL.

11. The magnetic resonance imaging system of claim 1, further comprising at least one computer readable medium comprising instructions, and wherein the at least one processor is configured to perform said acts of instructing, determining and generating by executing the instructions.

12. The magnetic resonance imaging system of claim 1, wherein the plurality of voxels of the 3D data set are arranged in a single layer of voxels, thereby representing a two-dimensional slice.

13. A computer-implemented method of metabolic imaging, the method comprising:
acquiring a deuterium-labeled three-dimensional (3D) data set of at least part of a subject;
determining, using at least one processor, a spectrum for each of a plurality of voxels of the 3D data set, at least a portion of the determined spectra comprising at least one peak corresponding to a deuterium-labeled molecule; and
generating, using the at least one processor, at least one metabolic image based at least in part on the spectra determined for the voxels in the 3D dataset.

14. The method of claim 13, wherein acquiring the deuterium-labeled 3D data set comprises instructing a magnetic resonance imager to apply one or more magnetic pulses to the at least part of the subject.

15. The method of claim 13, wherein the at least one peak corresponding to a deuterium-labeled molecule comprises a peak corresponding to ²H-labeled glucose, ²H-labeled lactate, ²H-labeled glutamine, or ²H-labeled glutamate.

16. The method of claim 13, further comprising quantifying, using the at least one processor, intensities of a plurality of peaks within the determined spectrum.

17. The method of claim 16, wherein the plurality of peaks include a peak that corresponds to ²H-labeled glucose and a peak that corresponds to ²H-labeled lactate.

18. The method of claim 13, wherein generating the at least one metabolic image comprises generating at least one color-coded image that reflects an amount of metabolic activity for one or more deuterium-labeled molecules at different voxel locations in a 3D volume.

19. The method of claim 13, further comprising determining, using the at least one processor, a glucose uptake rate and/or an acetate uptake rate.

20. At least one computer readable medium comprising instructions that, when executed by at least one processor, perform a method of metabolic imaging, the method comprising:
acquiring a deuterium-labeled three-dimensional (3D) data set of at least part of a subject;
determining, using at least one processor, a spectrum for each of a plurality of voxels of the 3D data set, at least a portion of the determined spectra comprising at least one peak corresponding to a deuterium-labeled molecule; and
generating, using the at least one processor, at least one metabolic image based at least in part on the spectra determined for the voxels in the 3D dataset.

21. The at least one computer readable medium of claim 20, wherein the at least one peak corresponding to a deuterium-labeled molecule comprises a peak corresponding to ²H-labeled glucose, ²H-labeled lactate, ²H-labeled glutamine, or ²H-labeled glutamate.

22. The at least one computer readable medium of claim 20, wherein generating the at least one metabolic image comprises generating at least one color-coded image that reflects an amount of metabolic activity for one or more deuterium-labeled molecules at different voxel locations in a 3D volume.
